(19) United States
(12) Patent Application Publication
(10) Pub. No.: US 2025/0369849 A1
(43) Pub. Date: Dec. 4, 2025

(54) METHOD FOR DETECTING PROCESSING DEGREE OF WHEAT FLOUR

(71) Applicant: Henan University of Technology, Zhengzhou City (CN)

(72) Inventors: Binghua SUN, Zhengzhou City (CN); Xiaoxi WANG, Zhengzhou City (CN); Sen MA, Zhengzhou City (CN); Zhenzhen MA, Zhengzhou City (CN); Erqi GUAN, Zhengzhou City (CN); Xiaojie QIAN, Zhengzhou City (CN)

(21) Appl. No.: 18/926,531

(22) Filed: Oct. 25, 2024

(30) Foreign Application Priority Data
Jun. 3, 2024 (CN) 202410711436.5

(51) Int. Cl. G01N 5/04; G01N 1/44; G01N 33/10

(52) U.S. Cl. CPC G01N 5/04; G01N 1/44; G01N 33/10

(57) ABSTRACT

A method for detecting a processing degree of wheat flour is provided, belonging to the technical field of analysis and detection. Thermogravimetric analysis of a wheat flour to be tested is conducted to obtain a pyrolysis characteristic curve, and then a correlation between characteristic values of a pyrolysis curve (including a pyrolysis residual mass fraction, a pyrolysis activation energy at 238° C. to 338° C., and a pyrolysis peak area at 290° C. to 320° C.) and key indicators of the processing degree of wheat flour (including ash content, bran speck content, and starch content) is established based on a pyrolysis kinetic equation and first derivative derivation. Weight values of three principal component indicators and their characteristic values are obtained by combining principal component analysis (PCA), such that a comprehensive evaluation function formula is constructed for the processing degree of wheat flour.

### CROSS REFERENCE TO RELATED APPLICATION

This patent application claims the benefit and priority of Chinese Patent Application No. 2024107114365 filed with the China National Intellectual Property Administration on Jun. 3, 2024, the disclosure of which is incorporated by reference herein in its entirety as part of the present application.

### TECHNICAL FIELD

The present disclosure belongs to the technical field of analysis and detection, and specifically relates to a method for detecting a processing degree of wheat flour.

### BACKGROUND

A wheat grain is composed of three parts from outside to inside: bran, endosperm, and embryo, and nutrients in the whole grain are unevenly distributed. The bran mainly contains cellulose, proteins, and minerals, while the endosperm mainly contains proteins and starch. The protein content increases progressively from the inside out, while the starch content gradually decreases from inside to outside. The existing wheat milling system generally involves layer-by-layer scraping from the inside out, grinding, and screening, followed by removal of bran and germ in the mid-to-late stages of flour extraction, with the endosperm particles serving as primary source flour extraction.

At present, there is a prevalent tendency in the grain processing industry to pursue wheat flour that is “refined, white, and fine”. The fundamental reason for this trend is that fine and white wheat flour exhibits excellent processing characteristics, such that flour products made therefrom have a desirable taste and can be sold at a higher price. However, the one-sided pursuit of “refined, white, and fine” also leads to excessive processing of wheat. At this time, a higher processing degree of wheat flour means less retention of bran such as an aleurone layer, resulting in more serious loss of nutrients such as cellulose, proteins, and minerals in the wheat bran. Therefore, in order to pursue a balance between nutrition, appearance, and taste, it is necessary to conduct research on appropriate wheat processing techniques. The precise control of processing degree of wheat flour is key to achieving moderate wheat processing. In GB/T1355-2021, the processing degree of wheat flour was defined as a degree of residual bran fragments in wheat flour, expressed by the size and number of bran specks. However, there is no unified standard to measure the “degree” of refined wheat flour so far. Most existing evaluation methods focus on a single indicator, such as flour yield, bran speck, color, particle size, and starch content, based on the cumulative ash curve. However, these indicators show poor synergy, unable to be directly quantified, leading to low applicability and accuracy of test results.

### SUMMARY

In view of this, the present disclosure provides a method for detecting a processing degree of wheat flour. In the present disclosure, the method is simple and practical, and can accurately and directly determine the processing degree of wheat flour.

To solve the above technical problem, the present disclosure provides a method for detecting a processing degree of wheat flour, including the following steps:
subjecting a wheat flour to be tested to thermogravimetric analysis, and then conducting first derivation on an obtained thermogravimetric analysis curve to obtain a first derivative thermogravimetric curve; where the thermogravimetric analysis is conducted at a pyrolysis temperature of 25° C. to 800° C.;
conducting calculations to obtain a pyrolysis residual mass fraction, a pyrolysis activation energy at 238° C. to 338° C., and a pyrolysis peak area at 290° C. to 320° C. according to the thermogravimetric analysis curve, a pyrolysis kinetic equation, and the first derivative thermogravimetric curve; and
conducting principal component analysis (PCA) with the pyrolysis residual mass fraction, the pyrolysis activation energy at 238° C. to 338° C., and the pyrolysis peak area at 290° C. to 320° C. as variables, identifying the pyrolysis residual mass fraction as a principal component 1, the pyrolysis activation energy at 238° C. to 338° C. as a principal component 2, and the pyrolysis peak area at 290° C. to 320° C. as a principal component 3, conducting calculations to obtain a, b, c, F1, F2, and F3, and then conducting calculations to obtain a comprehensive score F of the processing degree of wheat flour according to Formula 1, where a higher comprehensive score F indicates a higher processing degree of wheat flour;

$$F = aF 1 + bF 2 + cF 3 ; \qquad Formula 1$$

a, b, and c satisfy a+b+c=1; and
a represents weight of the principal component 1, b represents weight of the principal component 2, c represents weight of the principal component 3, F1 represents factor score of the principal component 1, F2 represents factor score of the principal component 2, and F3 represents factor score of the principal component 3.

In some embodiments, the thermogravimetric analysis has a heating rate of 8° C./min to 12° C./min.

In some embodiments, the thermogravimetric analysis is conducted under a protective atmosphere.

In some embodiments, the protective atmosphere includes a nitrogen atmosphere.

In some embodiments, the protective atmosphere has a flow rate of 80 mL/min to 120 mL/min.

In some embodiments, the wheat flour to be tested has a particle size of not more than 100 mesh.

In some embodiments, the thermogravimetric analysis has a sample loading amount of 5 mg to 7 mg.

In some embodiments, the pyrolysis kinetic equation is shown in Formula 2:

$$\frac{dx}{dt} = - {Ae}^{- \frac{E}{RT}} {χ}^{n} ; \qquad Formula 2$$

x represents relative mass fraction, in %; t represents time, in min; A represents pre-exponential factor, in min⁻¹; E represents activation energy, in kJ·mol⁻¹; T represents absolute temperature, in K; n represents reaction order; and R represents universal gas constant, in kJ·mol⁻¹·K⁻¹.

In some embodiments, x is calculated by Formula 3:

$$x = \frac{w - wf}{w 0 - wf} \times 100 \% ; \qquad Formula 3$$

w₀ represents initial mass of a sample, in mg; w_{f} represents final mass of the sample, in mg; and w represents mass of the sample at a certain temperature during heating, in mg.

The present disclosure provides a method for detecting a processing degree of wheat flour, including the following steps: subjecting a wheat flour to be tested to thermogravimetric analysis, and then conducting first derivation on an obtained thermogravimetric analysis curve to obtain a first derivative thermogravimetric curve; where the thermogravimetric analysis is conducted at a pyrolysis temperature of 25° C. to 800° C.; conducting calculations to obtain a pyrolysis residual mass fraction, a pyrolysis activation energy at 238° C. to 338° C., and a pyrolysis peak area at 290° C. to 320° C. according to the thermogravimetric analysis curve, a pyrolysis kinetic equation, and the first derivative thermogravimetric curve; and conducting PCA with the pyrolysis residual mass fraction, the pyrolysis activation energy at 238° C. to 338° C., and the pyrolysis peak area at 290° C. to 320° C. as variables, identifying the pyrolysis residual mass fraction as a principal component 1, the pyrolysis activation energy at 238° C. to 338° C. as a principal component 2, and the pyrolysis peak area at 290° C. to 320° C. as a principal component 3, conducting calculations to obtain a, b, c, F1, F2, and F3, and then conducting calculations to obtain a comprehensive score F of the processing degree of wheat flour according to Formula 1, where a higher comprehensive score F indicates a higher processing degree of wheat flour; F=aF1+bF2+cF3 Formula 1; a, b, and c satisfy a+b+c=1; and a represents weight of the principal component 1, b represents weight of the principal component 2, c represents weight of the principal component 3, F1 represents factor score of the principal component 1, F2 represents factor score of the principal component 2, and F3 represents factor score of the principal component 3. Thermogravimetric analysis of the wheat flour to be tested is conducted under a specific procedure to obtain a pyrolysis characteristic curve, and then correlation between characteristic values of a pyrolysis curve (including a pyrolysis residual mass fraction, a pyrolysis activation energy at 238° C. to 338° C., and a pyrolysis peak area at 290° C. to 320° C.) and key indicators of the processing degree of wheat flour (including ash content, bran speck content, and starch content) is established based on a pyrolysis kinetic equation and first derivative derivation. Weight values of three principal component indicators and their characteristic values are obtained by PCA, such that a comprehensive evaluation function formula is constructed for the processing degree of wheat flour. The method avoids the limitation of evaluating the processing degree of wheat flour by a single indicator. It provides a more accurate and intuitive comprehensive evaluation of processing degree of wheat flour, while allowing for the determination of contents of multiple nutritional components in wheat flour, which serves as a reference for the moderate and precise processing of wheat.

### BRIEF DESCRIPTION OF THE DRAWINGS

FIG. 1 shows a thermogravimetric analysis curve of the wheat flour in Example 1;

FIG. 2 shows a thermogravimetric analysis curve of the wheat flour in Example 2;

FIG. 3 shows a thermogravimetric analysis curve of the wheat flour in Example 3;

FIG. 4 shows a thermogravimetric analysis curve of the wheat flour in Example 4; and

FIG. 5 shows an F dot-line graph of the wheat flour samples with different flour yields.

### DETAILED DESCRIPTION OF THE EMBODIMENTS

The present disclosure provides a method for detecting a processing degree of wheat flour, including the following steps:
subjecting a wheat flour to be tested to thermogravimetric analysis, and then conducting first derivation on an obtained thermogravimetric analysis curve to obtain a first derivative thermogravimetric curve; where the thermogravimetric analysis is conducted at a pyrolysis temperature of 25° C. to 800° C.;
conducting calculations to obtain a pyrolysis residual mass fraction, a pyrolysis activation energy at 238° C. to 338° C., and a pyrolysis peak area at 290° C. to 320° C. according to the thermogravimetric analysis curve, a pyrolysis kinetic equation, and the first derivative thermogravimetric curve; and
conducting PCA with the pyrolysis residual mass fraction, the pyrolysis activation energy at 238° C. to 338° C., and the pyrolysis peak area at 290° C. to 320° C. as variables, identifying the pyrolysis residual mass fraction as a principal component 1, the pyrolysis activation energy at 238° C. to 338° C. as a principal component 2, and the pyrolysis peak area at 290° C. to 320° C. as a principal component 3, conducting calculations to obtain a, b, c, F1, F2, and F3, and then conducting calculations to obtain a comprehensive score F of the processing degree of wheat flour according to Formula 1, where a higher comprehensive score F indicates a higher processing degree of wheat flour;

$$F = aF 1 + bF 2 + cF 3 ; \qquad Formula 1$$

a, b, and c satisfy a+b+c=1; and
a represents weight of the principal component 1, b represents weight of the principal component 2, c represents weight of the principal component 3, F1 represents factor score of the principal component 1, F2 represents a factor score of the principal component 2, and F3 represents a factor score of the principal component 3.

In the present disclosure, the wheat flour to be tested is subjected to thermogravimetric analysis, and then an obtained thermogravimetric analysis curve is subjected to first derivation to obtain a first derivative thermogravimetric curve. The wheat flour to be tested has a particle size of preferably not more than 100 mesh. In some embodiments, the wheat flour to be tested is sieved using a sieve with an aperture of 100 mesh, and a resulting sieved material is taken to obtain the wheat flour to be tested with a particle size of not more than 100 mesh. Controlling the particle size of the wheat flour to be tested to not more than 100 mesh is conducive to the complete pyrolysis of the wheat flour and can eliminate an influence of the particle size factor on the processing degree of wheat flour.

In the present disclosure, the thermogravimetric analysis has a sample loading amount of preferably 5 mg to 7 mg, more preferably 6 mg. When the sample loading amount is less than 5 mg, the accuracy of information on components with less nutritional content such as protein in the wheat flour to be tested may be affected, and the thermogravimetric analysis curve may fluctuate greatly; when the sample loading amount is greater than 7 mg, it will affect the overflow of thermal decomposition gas products, resulting in deformation of the thermogravimetric analysis curve, poor resolution, and incomplete pyrolysis. The thermogravimetric analysis curve obtained by limiting the sample loading amount to 5 mg to 7 mg is the most stable and the information obtained is the most accurate.

In the present disclosure, the thermogravimetric analysis is conducted at a pyrolysis temperature of 25° C. to 800° C.; the thermogravimetric analysis has a heating rate of preferably 8° C./min to 12° C./min, more preferably 9° C./min to 10° C./min. The thermogravimetric analysis is preferably conducted under a protective atmosphere; the protective atmosphere preferably includes a nitrogen atmosphere. The protective atmosphere has a flow rate of preferably 80 mL/min to 120 mL/min, more preferably 100 mL/min to 110 mL/min.

In the present disclosure, the wheat flour to be tested is preferably placed in an alumina crucible and then placed on a sample holder of a thermogravimetric analyzer to allow pyrolysis. The thermogravimetric analyzer is preferably a TG 209 F3 thermogravimetric analyzer from NETZSCH, Germany.

In the present disclosure, there is no special requirement for a method of the first derivation, and the conventional method in the art can be used.

In the present disclosure, calculations are conducted to obtain a pyrolysis residual mass fraction, a pyrolysis activation energy at 238° C. to 338° C., and a pyrolysis peak area at 290° C. to 320° C. according to the thermogravimetric analysis curve, a pyrolysis kinetic equation, and the first derivative thermogravimetric curve. The pyrolysis kinetic equation is preferably shown in Formula 2:

$$\frac{dx}{dt} = - {Ae}^{- \frac{E}{RT}} {χ}^{n} ; \qquad Formula 2$$

x represents relative mass fraction, in %; t represents time, in min; A represents pre-exponential factor, in min⁻¹; E represents activation energy, in kJ·mol⁻¹; T represents absolute temperature, in K; n represents reaction order; and R represents universal gas constant, in kJ·mol⁻¹·K⁻¹.

In the present disclosure, x is preferably calculated by Formula 3:

$$x = \frac{w - wf}{w 0 - wf} \times 100 \% ; \qquad Formula 3$$

w₀ represents initial mass of the sample, in mg; w_{f} represents final mass of the sample, in mg; and w represents mass of the sample at a certain temperature during heating, in mg.

In the present disclosure, the pyrolysis residual mass fraction reflects the indicator of ash content in wheat flour, the pyrolysis activation energy at 238° C. to 338° C. reflects the indicator of bran speck content in wheat flour, and the pyrolysis peak area at 290° C. to 320° C. reflects the indicator of starch content in wheat flour. The ash content, bran speck content, starch content, and protein content in wheat flour are preferably detected according to the following methods: the ash content is detected in accordance with GB 5009.4-2016 “Determination of ash in food”; the bran speck content is detected in accordance with GB 27628-2011 “Determination of color and bran speck in wheat flour”; the starch content in wheat flour is determined by using Megazyme total starch kit according to the method in AACC76-31; the protein content is determined by the first method Kjeldahl nitrogen determination method in GB 5009.5-2016. Based on the test results, a correlation is established between the pyrolysis residual mass fraction, the pyrolysis activation energy at 238° C. to 338° C., the pyrolysis peak area at 290° C. to 320° C., and the pyrolysis peak area at 280° C. to 290° C. and the ash content, bran speck content, starch content, and protein content. The pyrolysis residual mass fraction and the evaluation indicator of processing degree of wheat flour—ash content are linearly correlated, with a correlation coefficient of 0.990 (y=0.6409x−12.703, R²=0.990); the pyrolysis activation energy at 238° C. to 338° C. and the evaluation indicator of processing degree of wheat flour-bran speck content are nonlinearly correlated in an exponential model, with a correlation coefficient of 0.956 (y=6E+13e^{-0.148x}, R²=0.956); and the pyrolysis peak area at 290° C. to 320° C. and the evaluation indicator of processing degree of wheat flour-starch content are linearly correlated, with a correlation coefficient of 0.94 (y=0.5536x+27.394, R²=0.940).

In the present disclosure, the pyrolysis peak area at 280° C. to 290° C. in the thermogravimetric analysis curve reflects the indicator of protein content in wheat flour. However, the correlation coefficient between the pyrolysis peak area at 280° C. to 290° C. and the protein content in wheat flour is 0.023 (y=0.0188x+13.146, R²=0.023). Due to the low correlation coefficient, the protein content is not used as the principal component for determining the processing degree of wheat flour.

In the present disclosure, after the pyrolysis residual mass fraction is obtained, the pyrolysis activation energy at 238° C. to 338° C., and the pyrolysis peak area at 290° C. to 320° C., PCA is conducted with the pyrolysis residual mass fraction, the pyrolysis activation energy at 238° C. to 338° C., and the pyrolysis peak area at 290° C. to 320° C. as variables, it is determined that the pyrolysis residual mass fraction is a principal component 1, the pyrolysis activation energy at 238° C. to 338° C. is a principal component 2, and the pyrolysis peak area at 290° C. to 320° C. is a principal component 3, calculations are conducted to obtain a, b, c, F1, F2, and F3, and then calculations are conducted to obtain a comprehensive score F of the processing degree of wheat flour according to Formula 1, where a higher comprehensive score F indicates a higher processing degree of wheat flour;

$$F = aF 1 + bF 2 + cF 3 ; \qquad Formula 1$$

a, b, and c satisfy a+b+c=1; and
a represents weight of the principal component 1, b represents weight of the principal component 2, c represents weight of the principal component 3, F1 represents factor score of the principal component 1, F2 represents factor score of the principal component 2, and F3 represents factor score of the principal component 3.

In the present disclosure, there is no special requirement for a method for the PCA, and the conventional method in the art can be used.

In order to further illustrate the present disclosure, the technical solutions provided by the present disclosure are described in detail below in connection with examples, but these examples should not be understood as limiting the claimed scope of the present disclosure.

### Example 1

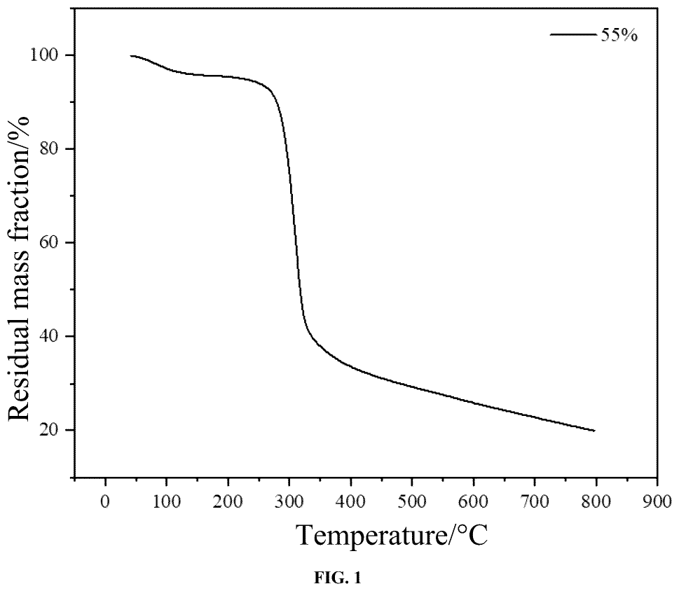

Commercially available wheat flour with a flour yield of 55% was used as a sample to be tested; the sample to be tested was passed through a 100-mesh sieve, and a sieved material was taken; 5 mg of the sieved material was placed in an alumina crucible and placed on the sample holder of a thermogravimetric analyzer (NETZSCH TG 209 F3, Germany) to allow thermogravimetric analysis, and a thermogravimetric analysis curve was obtained, as shown in FIG. 1; where conditions for thermogravimetric analysis included: a pyrolysis temperature of 25° C. to 800° C., a heating rate of 8° C./min, and a protective atmosphere of nitrogen atmosphere with a flow rate of 110 mL/min; first derivation of the thermogravimetric analysis curve was conducted to obtain a first derivative thermogravimetric curve.

The pyrolysis residual mass fraction was calculated from the thermogravimetric analysis curve to be 20.88%, the pyrolysis peak area at 290° C. to 320° C. was calculated from the first derivative thermogravimetric curve to be 90.36, and the pyrolysis activation energy at 238° C. to 338° C. was calculated from Formula 2 to be 185.80 kJ·mol⁻¹, where n was 2.84.

PCA was conducted with the pyrolysis residual mass fraction, the pyrolysis activation energy at 238° C. to 338° C., and the pyrolysis peak area at 290° C. to 320° C. as variables, and results showed that a was 0.62, b was 0.26, and c was 0.12; the score F1 of principal component 1 (pyrolysis residual mass fraction) was 3.33527, the score F2 of principal component 2 (pyrolysis activation energy at 238° C. to 338° C.) was 0.84089, and the score F3 of principal component 3 (peak area at 290° C. to 320° C.) was 1.12831; according to Formula 1, the comprehensive score of the processing degree of wheat flour was calculated as F=0.62×3.33527+0.26×0.84089+0.12×1.12831=2.42.

### Examples 2 to 4

The processing degree of the wheat flour to be tested was tested according to the method of Example 1, and the differences are shown in Table 1. The samples to be tested in Example 1 and Example 2 were all wheat flour with a flour yield of 55%, but were sourced from different manufacturers.

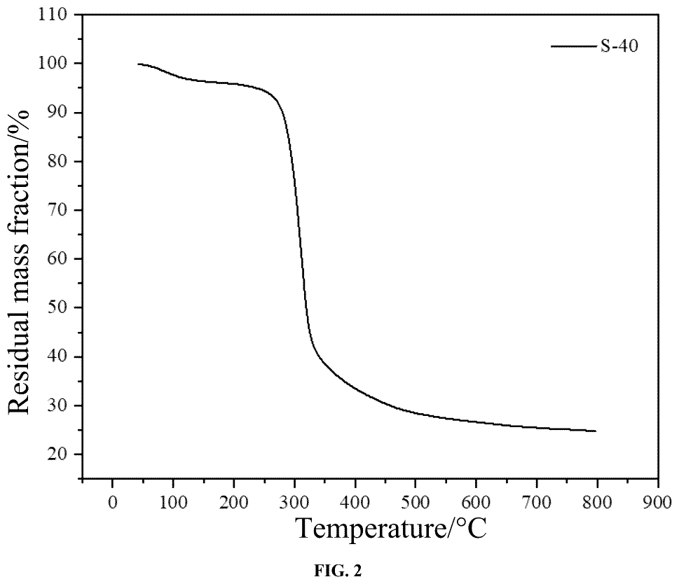

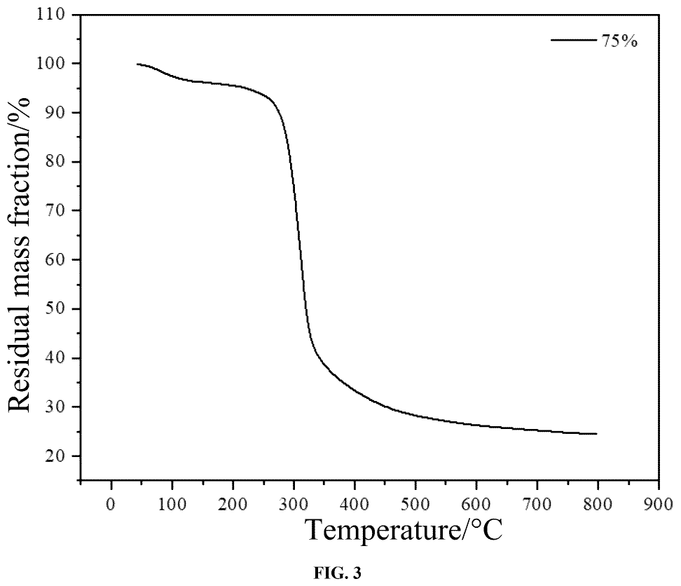

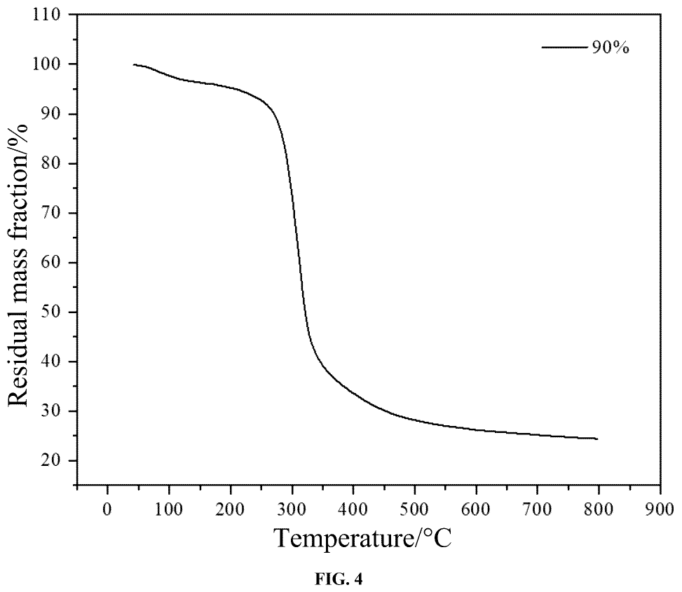

FIG. 2 to FIG. 4 are thermogravimetric analysis curves of Examples 2 to 4.

TABLE 1
Different parameters in detection methods of Examples 1 to 4

| Example | Example 1 | Example 2 | Example 3 | Example 4 |
|---|---|---|---|---|
| Flour yield (%) | 55 | 55 | 75 | 90 |
| Sample loading amount (mg) | 5 | 5 | 6 | 7 |
| Heating rate (° C./min) | 8 | 9 | 10 | 10 |
| Nitrogen flow rate (mL/min) | 110 | 100 | 100 | 110 |
| Residual mass fraction (%) | 20.88 | 20.44 | 21.63 | 22.45 |
| Pyrolysis peak area at 290° C. to 320° C. | 90.36 | 98.68 | 60.99 | 49.24 |
| Pyrolysis activation energy at | 185.80 | 203.85 | 166.17 | 153.36 |
| 238° C. to 338° C. (kJ · mol⁻¹) | | | | |
| a | 0.62 | 0.64 | 0.62 | 0.62 |
| b | 0.26 | 0.25 | 0.26 | 0.26 |
| c | 0.12 | 0.11 | 0.12 | 0.12 |
| F1 | 3.33527 | 1.053463 | −0.2792 | −2.3524 |
| F2 | 0.84089 | 0.361497 | −1.1101 | −1.2863 |
| F3 | 1.12831 | −0.07357 | 0.80375 | 0.81169 |
| F | 2.42 | 0.76 | −0.37 | −1.70 |

According to the data in Table 1, the F of Examples 1 and 2 was greater than the F of Examples 3 and 4. Namely, the wheat flour (flour yield of 55%) in Examples 1 and 2 had the highest processing degree, the wheat flour (flour yield of 75%) in Example 3 had a lower processing degree, and the wheat flour (flour yield of 90%) in Example 4 had the lowest processing degree. This result is consistent with the trend of processing degree of wheat flour determined by the flour yield of wheat flour in the examples.

The test results of Example 1 and Example 2 showed that the method provided by the present disclosure could more accurately analyze the processing degree of wheat flour with the same flour yield.

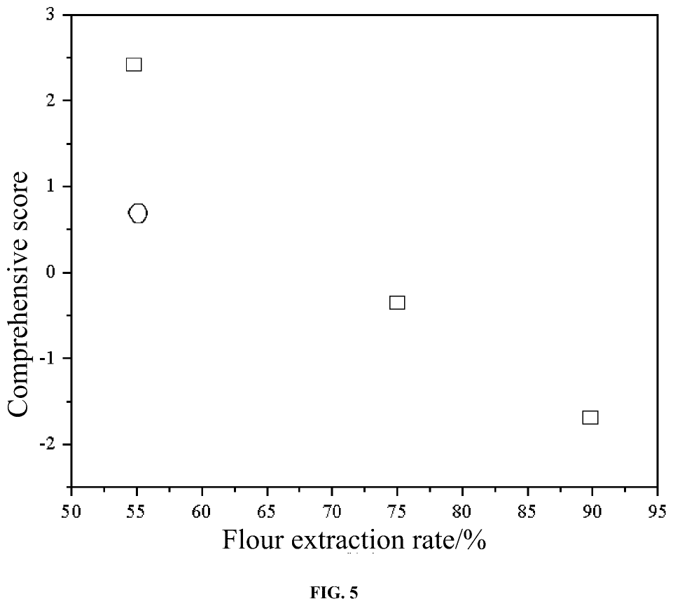

According to the data in Table 1, the F dot-line graph of the wheat flour samples to be tested with different flour yields (different examples) was drawn, as shown in FIG. 5. FIG. 5 indicates that the results of processing degree of wheat flour determined by F were roughly consistent with the results of processing degree of wheat flour determined by flour yield.

The starch content, bran speck content, protein content, and ash content in the wheat flour to be tested in Examples 1 to 4 were detected according to the following standards and recorded as measured values, respectively, and the results are listed in Table 2. The ash content was detected in accordance with GB 5009.4-2016 “Determination of ash in food”; the bran speck content was detected in accordance with GB 27628-2011 “Determination of color and bran speck in wheat flour”; the starch content in wheat flour was determined by using Megazyme total starch kit according to the method in AACC76-31; the protein content was determined by the first method Kjeldahl nitrogen determination method in GB 5009.5-2016.

The pyrolysis residual mass fraction, the pyrolysis peak area at 290° C. to 320° C., and the pyrolysis activation energy detected in the examples were calculated using a correlation equation to obtain the detection value of the ash content, the detection value of the bran speck content, the detection value of the starch content, and the detection value of the protein content. The results are listed in Table 2. The linear equation of pyrolysis residual mass fraction and ash content was: y=0.6409x−12.703, R²=0.990; the correlation equation between pyrolysis activation energy at 238° C. to 338° C. and bran speck content was: y=6E+13e^{-0.148x}, R²=0.956; the correlation equation between pyrolysis peak area at 290° C. to 320° C. and starch content was: y=0.5536x+27.394, R²=0.940; the correlation equation between pyrolysis peak area at 280° C. to 290° C. and protein content was: y=0.0188x+13.146, R²=0.023.

TABLE 2
Indicators of processing degree of wheat flour in Examples
1 to 4 obtained by testing according to existing standards

| | Starch content (%) | | Bran speck content (%₀₀) | | Ash content (%) | |
|---|---|---|---|---|---|---|
| | Measured | Detection | Measured | Detection | Measured | Detection |
| Example | value | value | value | value | value | value |
| Example | 74.52 | 77.41 | 30.02 | 68.47 | 0.595 | 0.67 |
| 1 | | | | | | |
| Example | 79.3 | 82.02 | 4.85 | 4.74 | 0.407 | 0.4 |
| 2 | | | | | | |
| Example | 64.13 | 61.16 | 1648.6 | 1251.43 | 1.12 | 1.16 |
| 3 | | | | | | |
| Example | 51.95 | 54.65 | 6513.73 | 8338.70 | 1.695 | 1.69 |
| 4 | | | | | | |

Table 2 shows that the starch content, bran speck content, and ash content in wheat flour could be accurately obtained according to the method provided in the present disclosure, such that the processing degree of wheat flour could be more comprehensively determined.

Although the above example has described the present disclosure in detail, it is only a part of, not all of, the embodiments of the present disclosure. Other embodiments may also be obtained by persons based on the example without creative efforts, and all of these embodiments shall fall within the protection scope of the present disclosure.

What is claimed is:

1. A method for detecting a processing degree of wheat flour, comprising the following steps:
subjecting a wheat flour to be tested to thermogravimetric analysis, and then conducting first derivation on an obtained thermogravimetric analysis curve to obtain a first derivative thermogravimetric curve; wherein the thermogravimetric analysis is conducted at a pyrolysis temperature of 25° C. to 800° C.;
conducting calculations to obtain a pyrolysis residual mass fraction, a pyrolysis activation energy at 238° C. to 338° C., and a pyrolysis peak area at 290° C. to 320° C. according to the thermogravimetric analysis curve, a pyrolysis kinetic equation, and the first derivative thermogravimetric curve; and
conducting principal component analysis (PCA) with the pyrolysis residual mass fraction, the pyrolysis activation energy at 238° C. to 338° C., and the pyrolysis peak area at 290° C. to 320° C. as variables, identifying the pyrolysis residual mass fraction as a principal component 1, the pyrolysis activation energy at 238° C. to 338° C. as a principal component 2, and the pyrolysis peak area at 290° C. to 320° C. as a principal component 3, conducting calculations to obtain a, b, c, F1, F2, and F3, and then conducting calculations to obtain a comprehensive score F of the processing degree of wheat flour according to Formula 1, wherein a higher comprehensive score F indicates a higher processing degree of wheat flour;
$$F = aF 1 + bF 2 + cF 3 ; \qquad Formula 1$$
a, b, and c satisfy a+b+c=1; and
a represents weight of the principal component 1, b represents weight of the principal component 2, c represents weight of the principal component 3, F1 represents factor score of the principal component 1, F2 represents factor score of the principal component 2, and F3 represents factor score of the principal component 3.

2. The method for detecting a processing degree of wheat flour according to claim 1, wherein the thermogravimetric analysis has a heating rate of 8° C./min to 12° C./min.

3. The method for detecting a processing degree of wheat flour according to claim 1, wherein the thermogravimetric analysis is conducted under a protective atmosphere.

4. The method for detecting a processing degree of wheat flour according to claim 3, wherein the protective atmosphere comprises a nitrogen atmosphere.

5. The method for detecting a processing degree of wheat flour according to claim 3, wherein the protective atmosphere has a flow rate of 80 mL/min to 120 mL/min.

6. The method for detecting a processing degree of wheat flour according to claim 5, wherein the protective atmosphere comprises a nitrogen atmosphere.

7. The method for detecting a processing degree of wheat flour according to claim 1, wherein the wheat flour to be tested has a particle size of not more than 100 mesh.

8. The method for detecting a processing degree of wheat flour according to claim 1, wherein the thermogravimetric analysis has a sample loading amount of 5 mg to 7 mg.

9. The method for detecting a processing degree of wheat flour according to claim 8, wherein the wheat flour to be tested has a particle size of not more than 100 mesh.

10. The method for detecting a processing degree of wheat flour according to claim 1, wherein the pyrolysis kinetic equation is shown in Formula 2:
$$\frac{dx}{dt} = - {Ae}^{- \frac{E}{RT}} {χ}^{n} ; \qquad Formula 2$$
x represents relative mass fraction, in %; t represents time, in min; A represents pre-exponential factor, in min−1; E represents activation energy, in kJ·mol−1; T represents absolute temperature, in K; n represents reaction order; and R represents universal gas constant, in kJ·mol−1·K−1.

11. The method for detecting a processing degree of wheat flour according to claim 8, wherein x is calculated by Formula 3:
$$x = \frac{w - wf}{w 0 - wf} \times 100 \% ; \qquad Formula 3$$
w0 represents initial mass of a sample, in mg; wf represents final mass of the sample, in mg; and w represents mass of the sample at a certain temperature during heating, in mg.
